(19) United States
(12) Patent Application Publication
(10) Pub. No.: US 2017/0112467 A1
(43) Pub. Date: Apr. 27, 2017

(54) RETENTION AND STABILIZATION OF ANATOMY FOR ULTRASOUND IMAGING

(71) Applicant: QT ULTRASOUND LLC, Novato, CA (US)

(72) Inventors: Mark Wayne LENOX, College Station, TX (US); Nasser Charles PIRSHAFIEY, Thousand Oaks, CA (US); Bilal Hameed MALIK, Novato, CA (US); John Charles KLOCK, Nicasio, CA (US)

(21) Appl. No.: 14/924,296

(22) Filed: Oct. 27, 2015

(51) Int. Cl. A61B 8/00; A61B 8/08

(52) U.S. Cl. CPC A61B 8/4209; A61B 8/0825; A61B 8/403; A61B 8/4281; A61B 8/587

(57) ABSTRACT

Systems and methods for the retention and stabilization of anatomy for ultrasound imaging are described. A system can include a vessel with a flexible membrane covering an opening, a fluid pressure sensor, a pump, and a pump controller for adding and removing fluid from the vessel until an optimum fluid pressure value or range of values is achieved. A method can include identifying an optimum fluid pressure range or value for retaining a specified body part in a stable position against a flexible membrane, monitoring the pressure of fluid in a vessel having the flexible membrane covering an opening, and directing a pump to add or remove fluid until the optimum fluid pressure value or range of values is achieved.

### BACKGROUND

Patient motion is the single largest cause of image artifacts in medical imaging systems. As these medical imaging systems are developed to achieve higher and higher resolutions, even very small patient motions can blur the imaging results. Physically restraining the patient can be very uncomfortable for the patient. For sensitive body parts, this can be a particularly difficult issue as people will tend to postpone or simply not do important medical imaging screening tests like mammography to avoid patient discomfort. Improving patient comfort can improve patient compliance with necessary or recommended medical imaging procedures as well as the effectiveness of the medical imaging by reducing the occurrence of image artifacts.

### BRIEF SUMMARY

Systems and methods for retention and stabilization of anatomy for ultrasound imaging are described. In some cases, body parts can be stabilized against a flexible membrane for patient comfort and minimizing imaging artifacts by using pressurized fluid that holds the body part against the flexible membrane during a medical imaging procedure.

A system can include a vessel with a flexible membrane covering an opening, a fluid pressure sensor, a pump, and a pump controller for adding and removing fluid from the vessel until an optimum fluid pressure value or range of values is achieved. A method can include identifying an optimum fluid pressure range or value for retaining a specified body part in a stable position against a flexible membrane, monitoring the pressure of fluid in a vessel having the flexible membrane covering an opening, and directing a pump to add or remove fluid until the optimum fluid pressure value or range of values is achieved.

Certain implementations include a computer readable storage medium with instructions stored thereon that, when executed, instruct a processor to identify an optimum fluid pressure value or range for retaining a specified body part in a stable position against a flexible membrane, monitor the pressure of fluid in a vessel having the flexible membrane covering an opening, and direct the pump to add or remove fluid until the optimum fluid pressure value or range is achieved. In some implementations, the storage medium can also store instructions to identify an approximate body part volume of fluid for a specified body part and direct a preliminary amount of fluid to be removed from a vessel based on the identified approximate body part volume of fluid.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

### BRIEF DESCRIPTION OF THE DRAWINGS

FIG. 1A-1C illustrate an example system configuration for the retention and stabilization of anatomy for ultrasound imaging.

FIGS. 2A and 2B illustrate yet another example system configuration for the retention and stabilization of anatomy for ultrasound imaging.

FIG. 3 shows a method of controlling the adding and removing of a fluid in a vessel for stable, comfort positioning of a body part against a flexible membrane.

FIG. 4 shows an example implementation of a method of controlling the adding and removing of a fluid in a vessel for stable, comfort positioning of a body part against a flexible membrane.

FIG. 5 shows a preliminary process that may be carried out for the stable, comfort positioning of a body part against a flexible membrane.

FIG. 6 illustrates an example implementation for a pump controller for stable, comfort positioning of a body part against a flexible membrane.

FIG. 7 illustrates components of an example computing system for a pump controller.

FIGS. 8A and 8B show ultrasound reflection images of a breast phantom made of agar with inclusions imaged with no retention (FIG. 8A) and with retention using a system for stabilization and retention of anatomy as described herein (FIG. 8B).

FIGS. 9A-9C illustrate a human breast imaged with no retention (FIG. 9A), with retention using a system for stabilization and retention of anatomy as described herein (FIG. 9B), and with water between flexible membrane and breast (FIG. 9C).

### DETAILED DISCLOSURE

Systems and methods for the retention and stabilization of anatomy for ultrasound imaging are described. The described system and methods can be a valuable tool in the stabilization of body parts for comfort positioning during medical imaging procedures and for minimizing the occurrence of imaging artifacts.

During a medical imaging procedure on a patient, the patient can rest their body part to be imaged against a flexible membrane that holds their body part in place using fluid in a vessel on the opposite side of the flexible membrane. Body parts that may be enclosed and retained in this manner by the flexible membrane include, but are not limited to, breast, hand, arm, leg, knee, shoulder, head, chest or the entire body. The arrangement and size of the membrane (and vessel) can be configured in a manner suitable for retention of a particular one or more body parts.

The comfort positioning in which the patient's body part is held in place can be accomplished by determining an optimum pressure fluid pressure range and controlling the amount of fluid in the vessel until the optimum fluid pressure range is achieved. In some cases, a software program is provided that receives, for example, from a practitioner or technician, an input indicating a specified body part that can include a body part type and a body part size. This input can be used to determine the optimum pressure fluid pressure range for the medical imaging procedure. The software program can be executed on a computer system that is in communication with a pump connected to the vessel to control the fluid pressure. In some cases, the system may be programmed to create a differential pressure condition in the vessel prior to engaging (inserting) the body part, which will result in a concave or convex (or other) shape in the membrane. The body part is then inserted in or onto the membrane. The pressure is then equalized increased or decreased to provide desired membrane contact and support, and to shape and locate the body part.

The computer system may communicate with the pump via a separate pump controller or communicate directly with the pump (e.g., as the pump controller). Through the pump controller (which may or may not be part of the computer system), the pump can be made to remove or add a certain amount of the fluid from or to the vessel until the optimum fluid pressure is achieved, or the desired volume of fluid is displaced. The pressure differential between the fluid in the vessel and the ambient pressure helps stabilize the body part in a comfortable position. Once the optimum fluid pressure differential is achieved, an ultrasound procedure can be initiated on the body part. The optimum fluid pressure differential can be maintained for the duration of the procedure for the comfort of the patient. In some cases, the operating software for the imaging can communicate with the pump controller. For example, the operating software for the imaging communicate to the pump controller an indication of a start or stop of the imaging. As another example, the pump controller can communicate to the operating software for the imaging an indication that optimum fluid pressure differential is achieved.

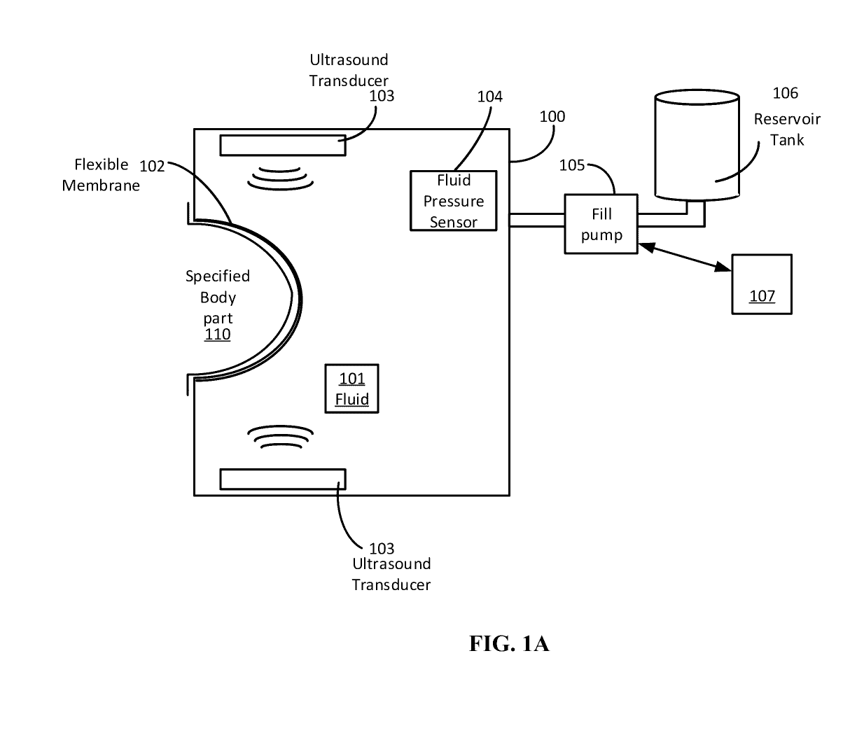

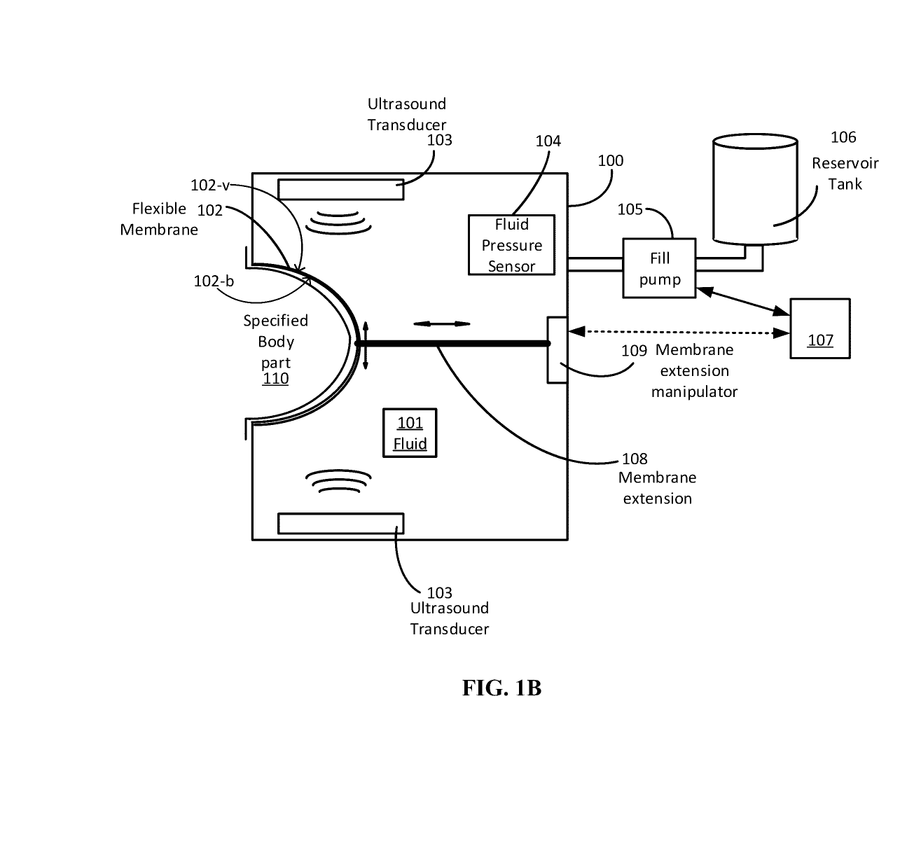

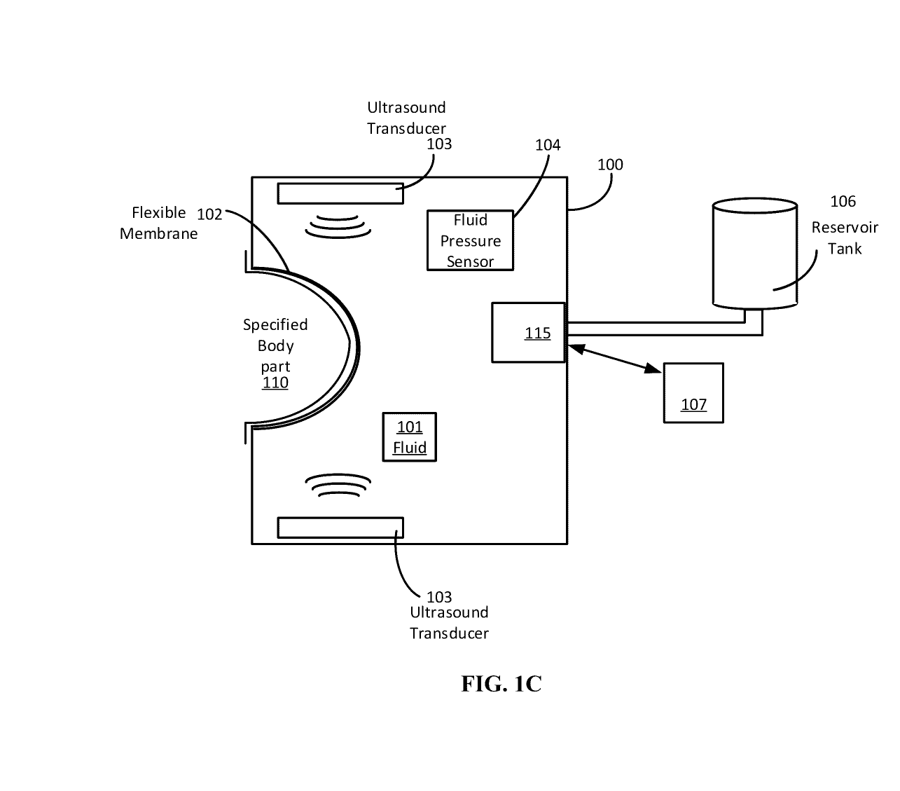

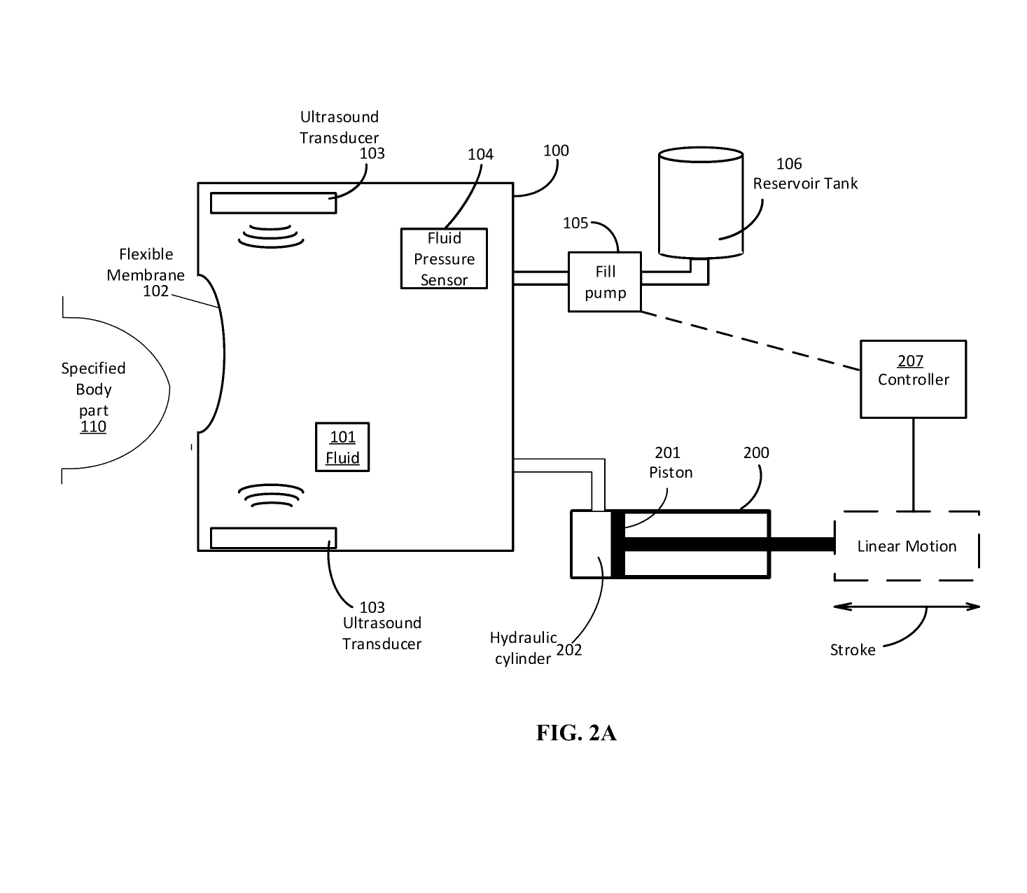

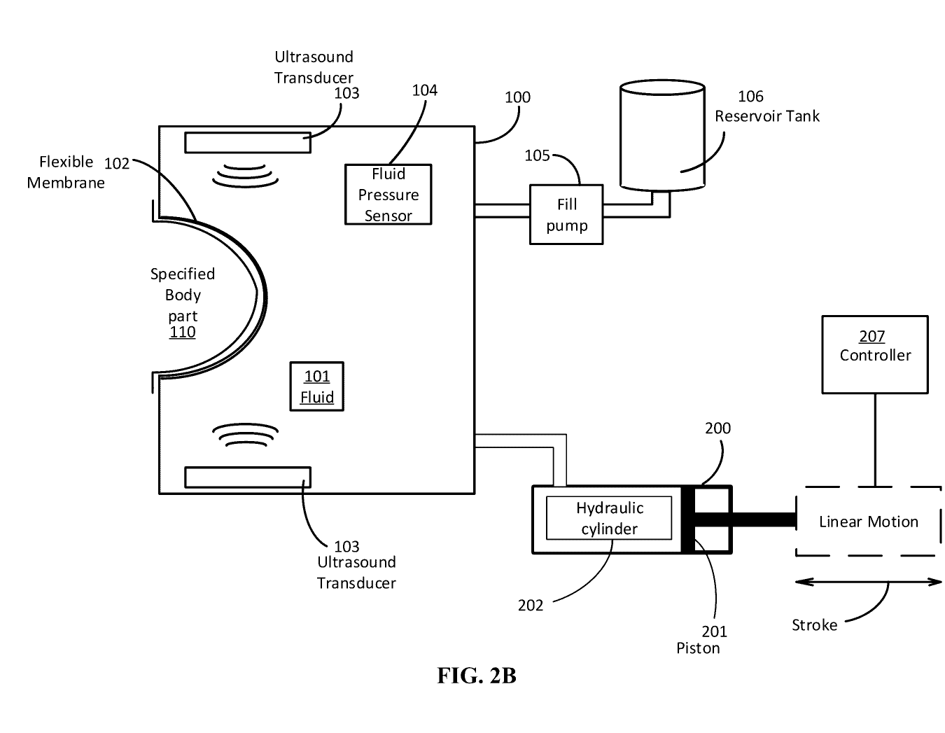

FIGS. 1A-1C illustrate example system configurations for the retention and stabilization of anatomy for ultrasound imaging; and FIGS. 2A and 2B illustrate yet another example system configuration for the retention and stabilization of anatomy for ultrasound imaging. Referring to FIG. 1A (and variously in FIGS. 1B, 1C, 2A, and 2B), a system for the retention and stabilization of anatomy for ultrasound imaging can include a vessel 100 for holding a fluid 101, a flexible membrane 102 covering an opening in the vessel 100, at least one ultrasound transducer 103 arranged around a region of the flexible membrane 102 for imaging a body part held by the flexible membrane 102, at least one fluid pressure sensor 104 for sensing the pressure of fluid in the vessel 100, and a pump 105 connected to a reservoir tank 106 and controlled by a pump controller 107.

The vessel 100 can be a hollow or concave device capable of holding a fluid. The vessel 100 can include various compartments and features; however at a minimum, the vessel contains a region for holding the fluid 101.

The fluid 101 can be water or saline. Of course, other fluids may be used. In many cases, the fluid is selected to be suitable for imaging.

The flexible membrane 102 covers an opening of the vessel 100. The flexible membrane 102 can be any fluid impermeable membrane such as, but not limited to, polymer membranes including latex or polyurethane. The particular attachment to the vessel (and permitted slack) can be any suitable means and arrangement selected based on body part to be imaged. The membrane 102 can be integrated in the vessel 100, or mounted to the vessel 100 for the purpose of removal to accommodate different body parts, sizes and shapes, and cleaning. The flexible membrane can be of a single piece of material that can enclose (or encase) and retain a body part or separate pieces of material that, in combination, can retain the body part.

In some embodiments, such as shown in FIG. 1B, the membrane 102 may optionally include a flexible or a non-flexile member(s) (membrane extension 108) that is attached to the vessel side of the membrane (side 102-v) and extends inside the vessel 100. The flexible or non-flexible member (membrane extension 108) can be pulled, pushed, or oriented to manipulate the location and shape of the membrane before or after the body part is engaged with the membrane (at body part side 102-b), for example, using a membrane extension manipulator 109. In some cases, the membrane extension manipulator 109 enables manual manipulation and adjustment of the membrane extension 108. In some cases, the membrane extension manipulator 109 can be controlled via programmed actuation of, for example, servos and motors. In some cases, the pump controller 107 (or computing system forming a part of the pump controller 107) or another controller or computing system can include functionality for controlling the membrane extension manipulator 109.

The at least one ultrasound transducer 103 is arranged so that a body part can be imaged while being held against the flexible membrane 102. The at least one ultrasound transducer 103 may be located inside the vessel 100.

In some cases, in addition to the at least one ultrasound transducer 103 inside the vessel 100, at least one transducer (not shown) can be incorporated outside the vessel 100 to extend the coverage of the image of the body part to the adjacent body part(s).

The ultrasound transducer(s) 103 (including any that may be incorporated outside the vessel 100) may be used to perform both reflection and transmission ultrasound methods. The reflection portion directs pulses of sound wave energy into tissues and receives the reflected energy from those pulses—hence it is referred to as “reflection ultrasound.” Detection of the sound pulse energies on the opposite side of a tissue after it has passed through the tissue is referred to as “transmission ultrasound.” Two ultrasound transducers 103 (or one receiver and one transmitter) can be located opposite each other with a specified body part held in place therebetween (e.g., body part 110) for performing transmission ultrasound. A single ultrasound transducer 103 can be used when performing reflection ultrasound. In some cases, the at least one ultrasound transducer 103 can be attached to a movable structure to enable the transducer to be rotated about the specified body part. In some cases, ultrasound transducers 103 are arranged about the vessel (directly or on a frame or other structure) so that the imaging can take place 360 degrees around the specified body part by selection of appropriate one(s) of the transducers to operate at a given time. In some cases, a combination of arrangement and physical rotation of the transducers may be utilized.

The specified body part 110 is kept in place against the flexible membrane 102 for the imaging by the pressure differential due in part to forces exerted by the fluid 101 in the vessel 100 against the flexible membrane 102, and therefore against the specified body part 110. The fluid pressure sensor 104 can be located inside the vessel 100 for detecting of the pressure of the fluid in the vessel. This data can be used by the system to control the amount of fluid in the vessel, and therefore, the amount of pressure that the fluid has against the flexible membrane and body part. In most cases, the measurements themselves are static measurements.

The fluid pressure sensor 104 can be any suitable sensor that sends a signal that can be interpreted by hardware or software to indicate the pressure in the vessel. The fluid pressure sensor 104 can include a transducer that converts a displacement caused by the pressure imposed by the fluid in the vessel to an electrical output (e.g., a voltage or current). The electrical output can be converted to a pressure measurement by hardware or software that is part of the sensor, part of a computing system for controlling retention and stabilization of anatomy, or a combination of both. Example types of transducers for pressure sensors include, but are not limited to, strain gage transducers, variable capacitance transducers, and piezoelectric transducers.

In some cases, the fluid pressure sensor 104 measures absolute pressure, which is the pressure of the fluid 101 inside the vessel 100 relative to a vacuum. In some cases, the fluid pressure sensor 104 measures gauge pressure, which is the pressure of the fluid 101 inside the vessel 100 relative to ambient atmospheric pressure. There may be some cases where the fluid pressure sensor 104 is configured for measuring relative pressure.

The pump 105 can be any suitable pump for adding or removing fluid from the vessel. The pump 105 includes a mechanical device that uses suction or pressure to raise or move fluid. In some implementations, such as illustrated in FIGS 1A and 1B, the pump 105 is located outside of the vessel 100. In other implementations, such as illustrated in FIG. 1C, a pump 115 is located inside the vessel 100. In either case, the pump 105, 115 may be a positive displacement pump, centrifugal pump, a piston pump, or axial flow pump. In addition, the pump 105, 115 may be considered submerged in the fluid it is pumping (whether in the reservoir or in the vessel) or placed external to the fluid (outside the reservoir or vessel).

A positive displacement pump is a mechanical device for moving fluid by trapping a fixed amount of fluid and displacing that amount. Example types of positive displacement pumps include, but are not limited to, gear, lobe, peristaltic, hydraulic cylinder, piston, bladder, diaphragm, and screw positive displacement pumps. A centrifugal pump uses a rotating impeller to discharge fluid from a central intake to a surrounding casing. An axial flow pump, or propeller pump, lifts fluid using an impellor arranged for axial discharge of fluid. A hydraulic cylinder displaces a set volume of fluid based on the position of the piston with in the cylinder. In this case, the position of the piston can be communicate with the controller. The electrical output of the piston location can be part of a computing system for controlling retention and stabilization of anatomy, or a combination of both. In another embodiment, a piston pump can have a single or multiple cylinders and displaces fluid by reciprocating its piston(s) in succession. A diagram or bladder pump can perform similarly.

In the case that the pump 105 (or the pump 115) is used to move fluid from the reservoir tank 106 to the vessel 100, a release valve or a second pump can be included either associated with the pump system or separately available in the vessel, but controllable by the pump controller 107 (and/or associated computing system), to remove, or release, fluid from the vessel.

For example, in the configuration illustrated in FIGS. 2A and 2B, a fill pump 105 is used to move fluid from the reservoir tank 106 to the vessel 100 and a second pump 200 is included for removing fluid 101 from the vessel. The second pump 200 can include a hydraulic cylinder 202 and piston 201 controlled by a controller 207. The controller 207 may also control the fill pump 105 so that fluid 101 may be controllable input to and removed from the vessel 100. As shown in FIG. 2A, the fill pump 105 can be used to fill the vessel 100. In some cases, some fluid may be removed by the second pump 200 before a specified body part 110 is positioned against the flexible membrane 102. Then, as shown in FIG. 2B, the controller 207 can be used to adjust the fluid 101 in the vessel 100 so that the body part 110 can be comfortably positioned (and held in place).

The fluid 101 removed from the vessel 100 may be collected in the reservoir tank 106 and reused by the pump 105 (or the pump 115) to add fluid to the vessel 100. In some cases, the pump 105, 115 can be configured to pull fluid from the reservoir tank 106 into the vessel and push fluid from inside the vessel to outside the vessel (and in some cases back in to the reservoir tank 106). The reservoir tank 106 can be a stand-alone tank or can be a chamber in the vessel 100 that is separate from that where the fluid 101 is provided.

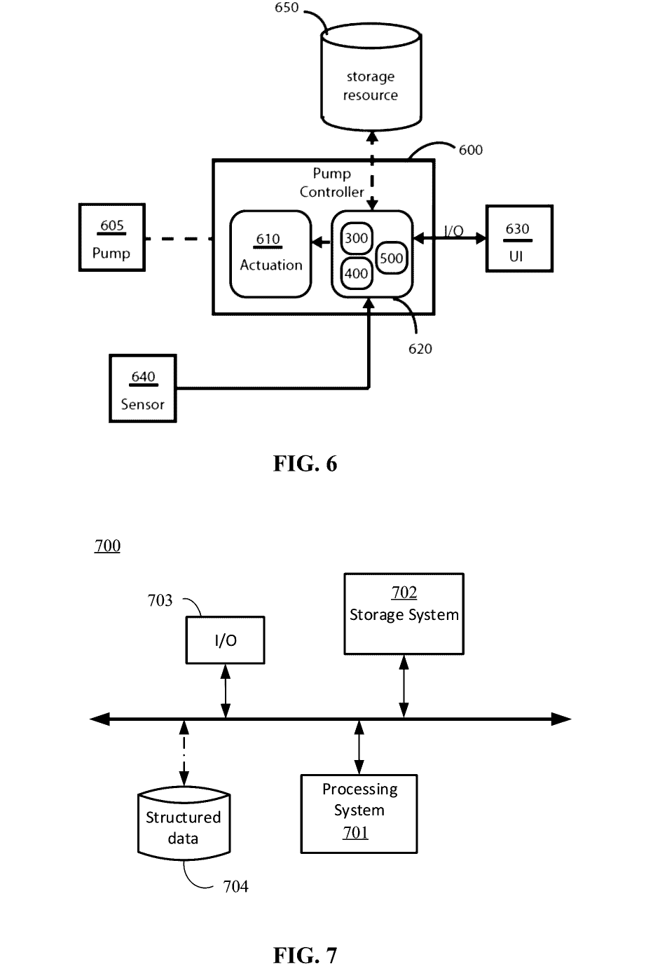

Pump controller 107, 207 controls the adding and removing of the fluid 101 from the vessel 100 for stable, comfort positioning of a body part against the flexible membrane 102. In certain embodiments, the pump controller 107, 207 can include components (hardware and/or software) that directly influence or direct the pump 105, 115 and/or 200 to pump out and/or pump in; and can include components (hardware and/or software) that perform processes to facilitate the stable, comfort positioning of a body part. Some aspects of these components may be carried out at the pump itself while other aspects may be carried out by a computing device such as described with respect to computing system 700 of FIG. 7. In certain embodiments, the pump controller can be implemented as illustrated in FIG. 6.

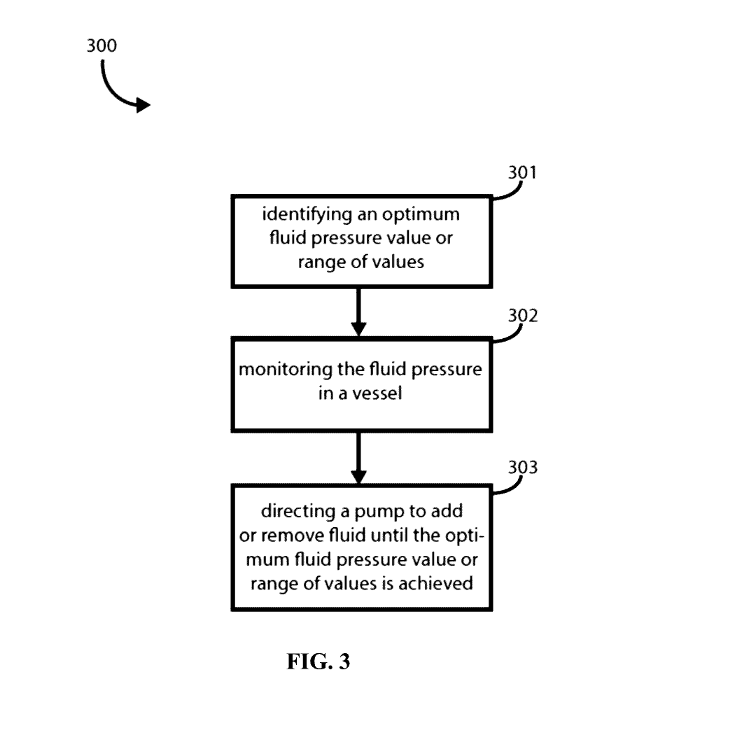

FIG. 3 shows a method of controlling the adding and removing of a fluid in a vessel for stable, comfort positioning of a body part against a flexible membrane. Referring to FIG. 3, a pump controller can carry out a process 300 that includes identifying an optimum fluid pressure value or range of values for a specified body part (301). The body part can be specified by one or more parameters received by the system that indicates information relative to the type of body part and also the size of the body part. In one implementation, the type and size of the body part can be determined by the system from an image by using an image recognition process. In another implementation, the type and size of the body part can be determined outside the system by any suitable technique (including visual inspection) and entered by a practitioner via the graphical user interface.

The optimum fluid pressure value or range of values may be identified by using data available to the system (which may be stored as structured data in a storage system that forms a part of or is available to the system by wired or wireless methods). The data available to the system may be quantitative and/or qualitative data originally obtained from studies on the range of effective pressure values on maintaining a particular body part (having a specific type and size) in place while maintaining the comfort of an individual. In one case, the optimum fluid pressure value or range of values is identified by performing a look-up using the one or more parameters received by the system to specify the body part.

The process 300 further includes monitoring the actual fluid pressure in a vessel (302); and directing a pump to add to or remove fluid from the vessel until the optimum fluid pressure value or range is achieved (303). The actual fluid pressure in the vessel can be monitored by monitoring an output of a fluid pressure sensor located in the vessel. Readings may be taken at specified intervals, periodically, or any other suitable times to achieve appropriate specificity. After analyzing the readings, the system can direct the pump through any suitable actuation methodologies (which depend, of course, on the type of pump used by the system).

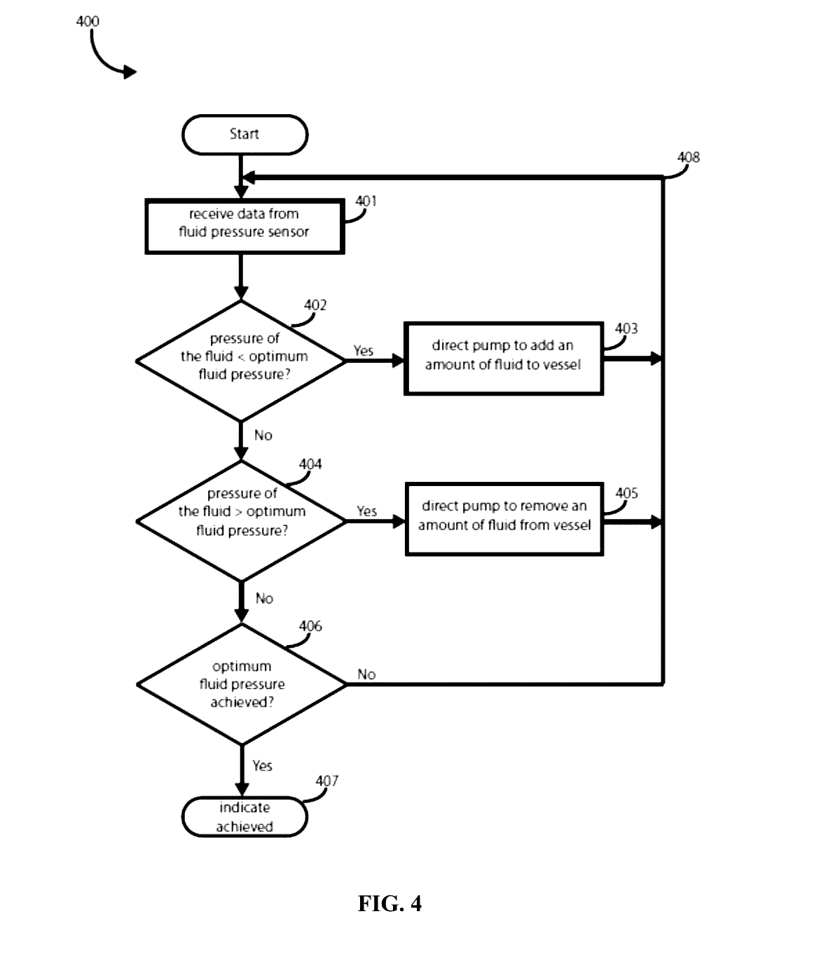

FIG. 4 shows an example implementation of a method of controlling the adding and removing of a fluid in a vessel for stable, comfort positioning of a body part against a flexible membrane. Referring to FIG. 4, a process 400 of monitoring actual fluid pressure in a vessel (e.g., operation 302 of FIG. 3) and directing a pump to add or remove fluid until optimum fluid pressure value or range is achieved (e.g., operation 303 of FIG. 3) can begin by receiving data from a fluid pressure sensor in the vessel (401). Using the received data and an identified optimum fluid pressure value or range of values (which may be obtained as described with respect to operation 301 of FIG. 3), a set of determining steps may be carried out.

As part of the determining steps, it can be determined whether a value from the fluid pressure measurement meets a criteria of being less than a value indicative of an optimum fluid pressure (402). If the value from the fluid pressure measurement is determined to be less than the value indicative of an optimum fluid pressure (e.g., through use of a software or hardware comparator), then the system directs the pump to add an amount of fluid to the vessel (403). After directing the pump to add the amount of fluid, the system can wait for receipt of data from the fluid pressure sensor or otherwise return to beginning of process 400 to repeat (408). The amount of fluid added to the vessel may be a set amount of fluid or set amount of time (that is set by the physical constraints of the system or by a program input or preset). In some cases, the amount of fluid added to the vessel may depend on an additional computation and use a value indicative of the difference between the two pressure values. For example, the value indicative of the difference between the two pressure values can be correlated to an amount of fluid (and have a mathematical relationship); and that amount of fluid can be directed to be added. In yet another case, the pump is directed to add fluid until the data from the pressure sensor indicates that the pressure is within the optimum range of values (or met a criteria of an optimum value).

If the pressure is determined to not be less than the optimum fluid pressure range, it can be determined whether a value from the fluid pressure measurement meets a criteria of being greater than the value indicative of the optimum fluid pressure (404). If the value from the fluid pressure measurement is determined to be greater than the value indicative of the optimum fluid pressure (e.g., through use of a software or hardware comparator), then the pump is directed to remove an amount of fluid to the vessel (405). After directing the pump to remove the amount of fluid, the system can wait for receipt of data from the fluid pressure sensor or otherwise return to beginning of process 400 to repeat (408).

As with that described with respect to operation 403, the amount of fluid removed from the vessel during operation 405 may be a set amount of fluid or set amount of time (that is set by the physical constraints of the system or by a program input or preset). In some cases, the amount of fluid removed from the vessel may depend on an additional computation and use a value indicative of the difference between the two pressure values. For example, the value indicative of the difference between the two pressure values can be correlated to an amount of fluid (and have a mathematical relationship); and that amount of fluid can be directed to be removed. In yet another case, the pump is directed to remove fluid until the data from the pressure sensor indicates that the pressure is within the optimum range of values (or met a criteria of an optimum value).

The pump used to add fluid and the pump used to remove fluid may be part of a same pump system, may be different pumps, or may be different types of pumps.

As further shown in process 400, it can be determined whether the optimum fluid pressure value or range has been achieved (406). If the optimum fluid pressure value or range has been achieved, for example by determining that the data from the pressure sensor indicates that the fluid pressure is within the optimum range of values or at the optimum value, then the system can indicate (explicitly or implicitly) that the optimum value has been achieved (407). This may indicate the end of the process. If the optimum pressure is determined to have not yet been achieved, the determining steps (along with possible directing of adding or removing fluid) can repeat (408). In some cases, operation 406 for the determination whether the optimum fluid pressure value or range has been achieved may not be an active determining step. Instead, the result of the determining that the pressure is not less than or greater than the optimum pressure value.

It should be understood that although the determining steps are shown and described in a certain order, these steps may be carried out in any suitable order or even in parallel. For example, in some cases, the determining steps begin with determining whether the value from the sensor meets the criteria of the optimum fluid pressure and if not, then it is determined whether fluid should be added or removed (e.g., whether the value from the sensor is less than or greater than the optimum fluid pressure) before repeating the determining steps again.

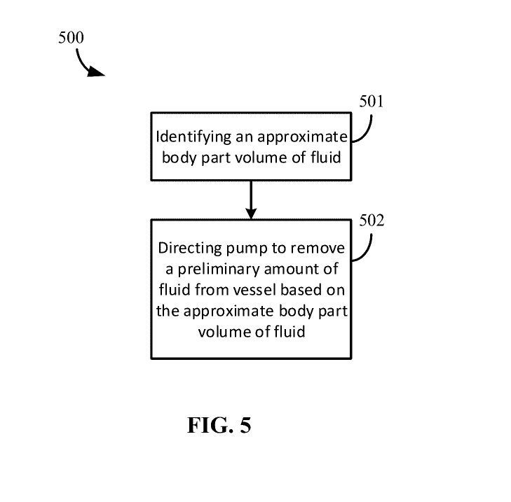

FIG. 5 shows a preliminary process that may be carried out for the stable, comfort positioning of a body part against a flexible membrane. Referring to FIG. 5, a preliminary process 500 can be carried out before monitoring the fluid pressure in a vessel and directing a pump to add or remove fluid. That is, when information is received regarding a specified body part that will be imaged, an approximate body part volume of fluid can be identified (501); and the pump directed to remove a preliminary amount of fluid from a vessel filled with fluid based on the approximate body part volume of fluid (502). This removal can facilitate the positioning of a patient's body part and/or speed up the process of retaining the body part in a stable position.

The body part can be specified by one or more parameters received by the system that indicate information relative to the type of body part and also the size of the body part. In one implementation, the type and size of the body part can be determined by the system from an image by using an image recognition process. In another implementation, the type and size of the body part can be determined outside the system by any suitable technique (including visual inspection) and entered by a practitioner via the graphical user interface.

In some embodiments, the approximate body part volume of fluid may be identified during operation 501 from a database of approximate body part volumes of fluid, for example, by performing a look-up in the database using the type and size information received by the system. In another embodiment, calculations can be carried out in response to receiving the information indicative of the type and size information to identify the corresponding amount of fluid that would be approximately equivalent to that specified body part's fluid volume. This corresponding amount of fluid may be considered equal to the preliminary amount of fluid that will be removed from the vessel to make room for the body part to extend into the vessel. In some cases, the preliminary amount of fluid indicated to be removed can be the equivalent volume (equivalent to the specified body part's fluid volume) plus a certain amount. Some of the information regarding the body part's fluid volume may be used to identify an optimum fluid pressure in a subsequent step (e.g., step 301 of process 300).

In some cases, after the preliminary amount of fluid is removed, the comfort positioning can be manually performed by a practitioner adjusting the controls of the pump. In some cases, after both the preliminary amount of fluid is removed and the optimal fluid pressure for the stable, comfort positioning of the body part is identified, the system can perform the processes of monitoring the fluid pressure and directing the pump to add or remove fluid until the optimal fluid pressure is achieved such as described with respect to operations 302 and 303 of FIG. 3 (and example implementation described with respect to FIG. 4).

FIG. 6 illustrates an example implementation for a pump controller for stable, comfort positioning of a body part against a flexible membrane. Referring to FIG. 6, a pump controller 600 can include an actuation component 610 for directing signals to a pump 605 (which may be implemented, for example, as pump 105, pump 115, and/or pump 200) based on output of a control component 620. The control component 620 can take at least user-input (e.g., via a user interface 630) and data from a sensor 640 (e.g., from fluid pressure sensor 104) as input. In some cases, the control component 620 can direct communications with a storage resource 650 that may be local or remote from the pump controller 600 (e.g., to obtain body part related data such as approximate body part volume of fluid and optimum fluid pressure value(s)).

The actuation component 610 can enable reverse (pump out) and/or direct (pump in) acting. The control component 620 may perform processes such as process 300 described with respect to FIG. 3, process 400 described with respect to FIG. 4, and/or process 500 described with respect to FIG. 5.

The functionality provided by the actuation component 610 (and in some cases aspects of the control component 620) may be in the form of logic implemented in hardware such as a field programmable gate array (FPGA) or an application specific integrated circuit (ASIC). In other embodiments, this functionality may be implemented at least in part in software stored on a storage medium and executed by a processing system such as described with respect to the computing system 700 of FIG. 7. In some cases, both the actuation component 610 and the control component 620 are implemented by this computing system. In other cases, certain aspects or elements of the two components (particularly the actuation component 610) are implemented by hardware and/or software (stored in a storage medium) located at or near the pump in order to actuate (or cause actuation of) the pump.

When certain aspects or elements of the components are located separately from other aspects, elements, or components of the pump controller, any of a variety communication mechanisms may be used to communicate between the aspects, elements, or components, including, but not limited to, wired and wireless connections enabling communication via any suitable communication protocol and over any suitable communication network (e.g., cellular, Bluetooth, near-field, Wi-Fi, optical fiber, cable, etc.).

FIG. 7 illustrates components of an example computing system for a pump controller as described herein. The computing system may be embodied as a desktop computer, lap top, tablet, server, mobile device, appliance, or other computing device or combination thereof. The computing system 700 can include a processing system 701 and a storage system 702.

Processing system 701 can include general purpose central processing units, application specific processors, and logic devices, as well as any other type of processing device, combinations, or variations thereof. Processing system 701 processes data according to instructions stored on the storage system 702. In certain implementations, storage system can store instructions for performing any of processes 300, 400, and 500, or portions thereof, as well as any instructions for providing actuation component 610 and control component 620.

Storage system 702 includes any computer readable storage media readable by the processing system 701 and capable of storing software. Storage system 702 may include volatile and nonvolatile, removable and non-removable media implemented in any method or technology for storage of information. Examples of storage media include random access memory (RAM), read only memory (ROM), magnetic disks, optical disks, CDs, DVDs, flash memory, solid state memory, phase change memory, or any other suitable storage media. Certain implementations may involve either or both virtual memory and non-virtual memory. In no case do storage media consist of a propagated signal or carrier wave. In addition to storage media, in some implementations, storage system 702 may also include communication media over which software may be communicated internally or externally.

Storage system 702 may be implemented as a single storage device but may also be implemented across multiple storage devices or sub-systems co-located or distributed relative to each other. Storage system 702 may include additional elements, such as a controller, capable of communicating with processing system 701.

System 700 can also include Input/Output (I/O) devices 703 such as a keyboard, mouse, display, touchscreen, network card, communication interface, or other I/O device.

A storage resource 704 can be included as part of computing system 700 or simply be accessible by computing system 700 (e.g., via a communication interface). In either case, storage resource 704 may be coupled to the system via wired or wireless connections. Storage resource 704 may store databases or other structured data containing volumes for specified body parts and sizes and optimum fluid pressure values or ranges of values. It should be understood the any computing device implementing the described system may have additional features or functionality and is not limited to the configurations described herein.

Examples illustrating the effectiveness of retaining a body part for ultrasound imaging using a flexible membrane are described below.

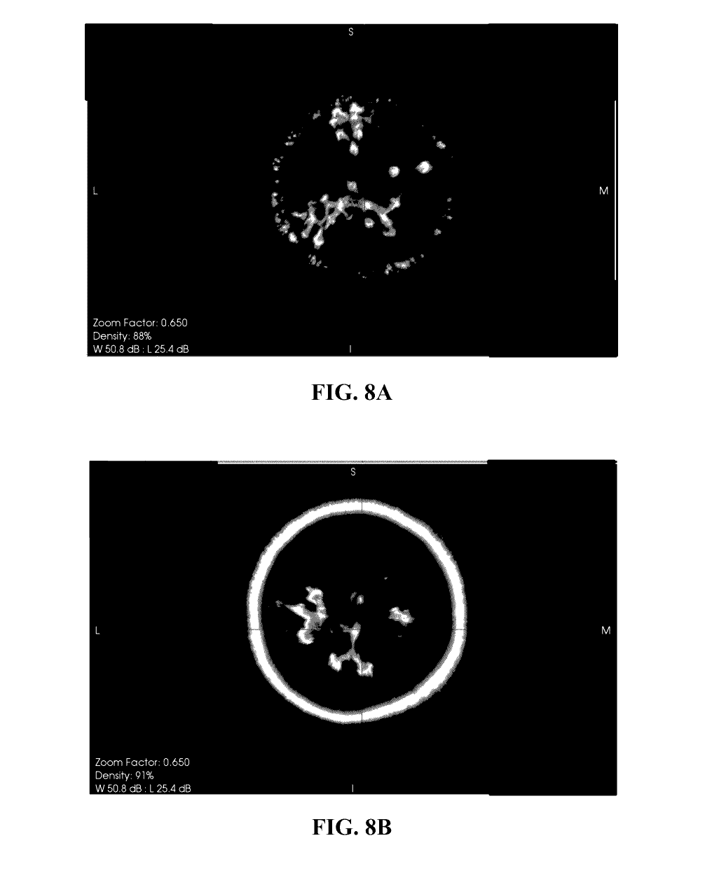

FIGS. 8A and 8B show ultrasound reflection images of a breast phantom made of agar with inclusions imaged with no retention (FIG. 8A) and with retention using a system for stabilization and retention of anatomy as described herein (FIG. 8B). The flexible membrane used in the imaging example of FIG. 8B is a latex membrane. When comparing the images of FIGS. 8A and 8B, it can be seen that additional clarity is available by using the system.

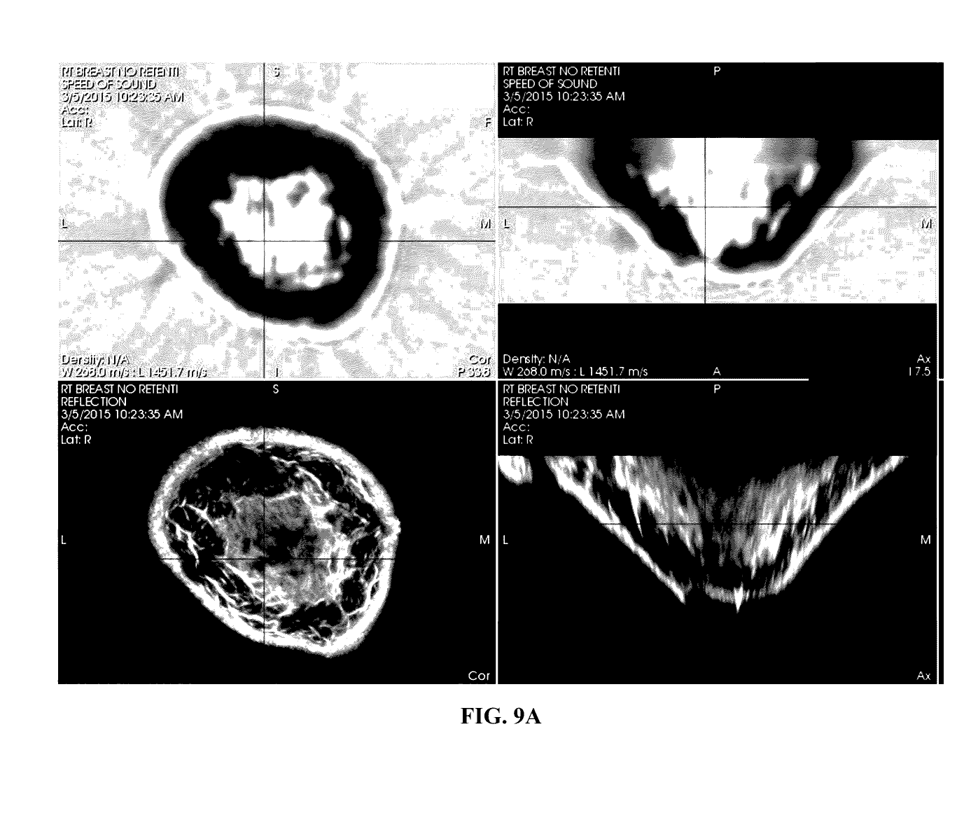

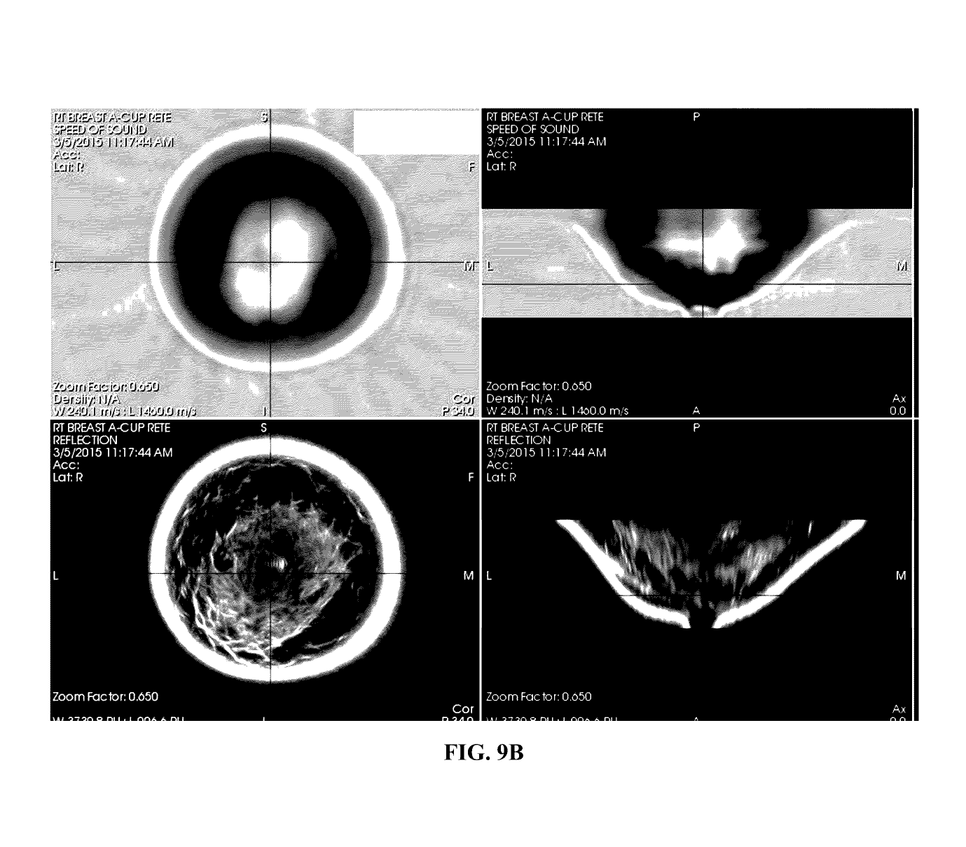

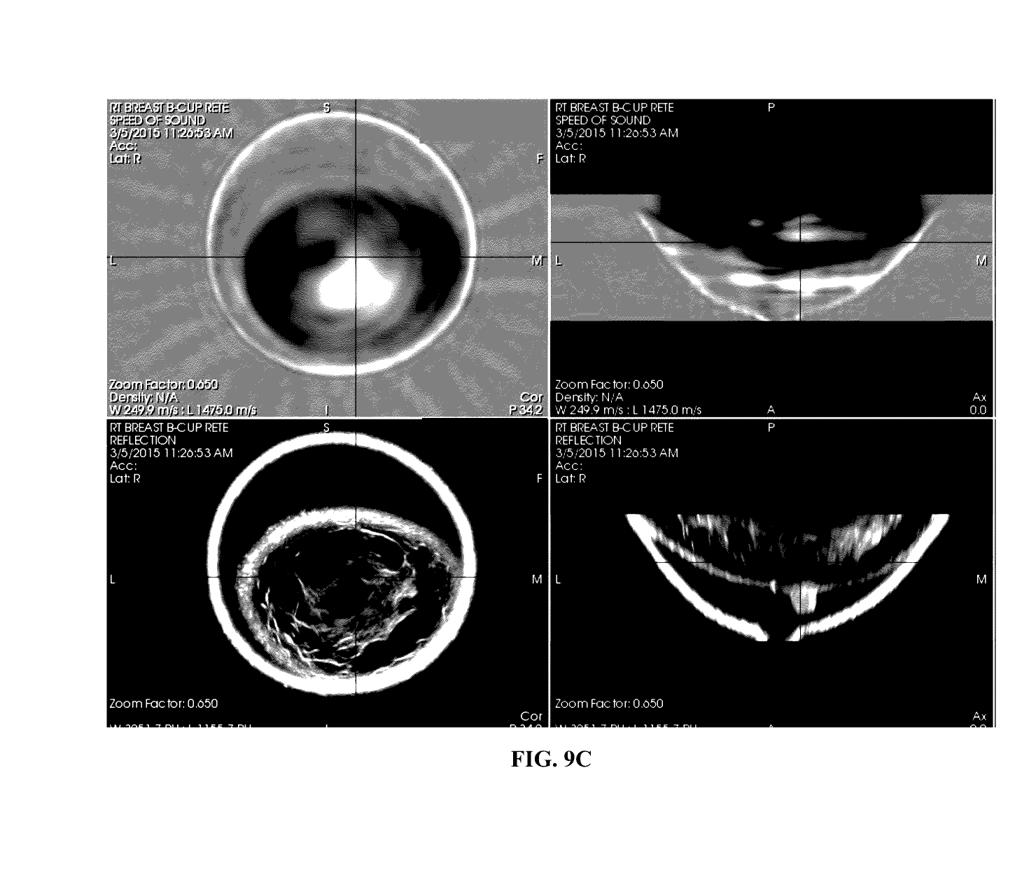

FIGS. 9A-9C illustrate a human breast imaged with no retention (FIG. 9A), with retention using a system for stabilization and retention of anatomy as described herein (FIG. 9B), and with water between flexible membrane and breast (FIG. 9C). The flexible membrane used in the imaging examples of FIGS. 9B and 9C is a latex membrane. The images include speed of sound in coronal plane (top left), speed of sound in axial plane (top right), reflection in coronal plane (bottom left), and reflection in axial plane (bottom right). Again, it can be seen that imaging using the described system can maintain stable arrangement of the breast.

It should be understood that the examples and embodiments described herein are for illustrative purposes only and that various modifications or changes in light thereof will be suggested to persons skilled in the art and are to be included within the spirit and purview of this application.

What is claimed is:

1. A system comprising:
a vessel for holding a fluid;
a flexible membrane, wherein the flexible membrane covers an opening of the vessel;
at least one ultrasound transducer;
at least one fluid pressure sensor for sensing pressure of the fluid inside the vessel;
a pump for adding or removing the fluid from the vessel; and
a pump controller controlling the adding and removing of the fluid for stable, comfort positioning of a body part against the flexible membrane.

2. The system of claim 1, wherein the pump controller comprises:
a processing system; and
at least one storage medium having instructions stored thereon that, when executed by the processing system, direct the processing system to at least:
identify an optimum fluid pressure value or range of values for maintaining a specified body part in a stable position against the flexible membrane;
monitor an output of the at least one fluid pressure sensor; and
direct the pump to remove the fluid from or add the fluid to the vessel based on the output of the at least one fluid pressure sensor until the optimum fluid pressure value or range is achieved.

3. The system of claim 2, wherein the instructions further direct the processing system to maintain the optimum fluid pressure value or range during an imaging process of the specified body part using the at least one ultrasound transducer.

4. The system of claim 2, wherein the instructions further direct the processing system to:
identify, based on at least one parameter associated with the specified body part, the optimum fluid pressure value or range of values for maintaining the body part in a stable position against the flexible membrane.

5. The system of claim 4, wherein the instructions that direct the processing system to identify the optimum fluid pressure value or range of values comprise instructions that direct the processing system to perform a look-up process on structured data stored on a device accessible by the processing system.

6. The system of claim 5, wherein the at least one parameter associated with the specified body part comprises at least one parameter indicative of a type and a size of the specified body part.

7. The system of claim 1, wherein the pump controller comprises:
a processing system; and
at least one storage medium having instructions stored thereon that, when executed by the processing system, direct the processing system to:
in response to receiving one or more parameters indicative of a type and a size of a specified body part, identify an approximate body part volume of fluid from a database storing volumes of known sizes of body parts; and
in response to receiving an indication to initialize the vessel for receiving the specified body part, direct the pump to remove the approximate body part volume of fluid from the vessel.

8. The system of claim 1 wherein the fluid is water or saline.

9. The system of claim 1 wherein the flexible membrane is a polymer based membrane.

10. The system of claim 1 wherein the fluid pressure sensor is located inside of the vessel.

11. The system of claim 1 wherein the pump is a positive displacement pump.

12. The system of claim 1 wherein the pump is located inside of the vessel.

13. The system of claim 1, further comprising at least one reservoir tank located outside of the vessel.

14. A method comprising:
identifying an optimum fluid pressure value or range of values for maintaining a specified body part in a stable position against a flexible membrane separating the specified body part from a fluid in a vessel;
monitoring a pressure of the fluid; and
directing a pump to remove the fluid from or add the fluid to the vessel based on the pressure of the fluid until the optimum fluid pressure range is achieved.

15. The method of claim 14, wherein directing the pump to remove the fluid from or add the fluid to the vessel based on the pressure of the fluid comprises:
determining whether the pressure is above or below the optimum fluid pressure value or range; and
in response to determining that the pressure is above the optimum fluid pressure value or range, directing the pump to remove an amount of fluid from the vessel;
in response to determining that the pressure is below the optimum fluid pressure value or range, directing the pump to add an amount of fluid to the vessel.

16. The method of claim 14, further comprising maintaining the optimum fluid pressure value or range during an imaging process of the specified body part.

17. The method of claim 14, further comprising identifying an approximate body part volume of fluid based on one or more parameters indicative of a type and a size of the specified body part and directing the pump to remove a preliminary amount of fluid based on the approximate body part volume of fluid.

18. The method of claim 14, further comprising initiating an ultrasound of the specified body part while the specified body part is maintained in the stable position by the fluid providing the optimum fluid pressure against the flexible membrane.

19. One or more computer-readable storage media having instructions stored thereon that, when executed by a processing system, direct the processing system to:
in response to receiving one or more parameters indicative of a type and a size of a specified body part, identify an approximate body part volume of fluid from a database storing volumes of known sizes of body parts;
in response to receiving an indication to initialize a vessel for receiving the specified body part direct a pump to remove the approximate body part volume of fluid from a total amount of fluid the vessel;
identify an optimum fluid pressure value or range of values for maintaining the specified body part in a stable position against a flexible membrane separating the specified body part from the fluid in the vessel; and
direct a pump to remove the fluid from or add the fluid to the vessel based on a detected pressure of the fluid in the vessel until the optimum fluid pressure value or range is achieved.

20. The media of claim 19, wherein the instructions that direct the processing system to direct the pump to remove the fluid from or add the fluid to the vessel based on the pressure of the fluid, direct the processing system to:
determine whether the detected pressure is above or below the optimum fluid pressure value or range; and
in response to determining that the detected pressure is above the optimum fluid pressure value or range, direct the pump to remove an amount of fluid from the vessel;
in response to determining that the detected pressure is below the optimum fluid pressure value or range, direct the pump to add an amount of fluid to the vessel.
